(19) United States
(12) Patent Application Publication
(10) Pub. No.: US 2020/0275865 A1
(43) Pub. Date: Sep. 3, 2020

(54) ELECTRONIC DEVICE AND CONTROL METHOD THEREFOR

(71) Applicant: SAMSUNG ELECTRONICS CO., LTD., Suwon-si, Gyeonggi-do (KR)

(72) Inventors: Hyoung-seon CHOI, Seoul (KR); Seong-je CHO, Suwon-si, Gyeonggi-do (KR); Jin-hong MIN, Suwon-si, Gyeonggi-do (KR); Young-jae OH, Suwon-si, Gyeonggi-do (KR); Kyoung-jin MOON, Suwon-si, Gyeonggi-do (KR); Chul-ho CHO, Yongin-si, Gyeonggi-do (KR)

(21) Appl. No.: 16/494,958

(22) PCT Filed: Mar. 19, 2018
(86) PCT No.: PCT/KR2018/003170

§ 371 Date: Sep. 17, 2019

Related U.S. Application Data
(60) Provisional application No. 62/472,656, filed on Mar. 17, 2017.

(30) Foreign Application Priority Data
May 24, 2017 (KR) 10-2017-0064263

(51) Int. Cl. A61B 5/145; A61B 5/01; A61B 5/1486; A61B 5/00

(52) U.S. Cl. CPC A61B 5/14532; A61B 5/01; A61B 2560/0252; A61B 5/7275; A61B 5/0008; A61B 5/14865

(57) ABSTRACT

An electronic device and a control method therefor are disclosed. The electronic device control method according to the present invention comprises the steps of: receiving blood glucose information and skin temperature information from a blood glucose meter; obtaining external temperature information of a region in which a user wearing the blood glucose meter is located; determining, by using the skin temperature information and the external temperature information, the predictive temperature of an inner skin part at which an enzyme sensor of the blood glucose meter is located; correcting the blood glucose information on the basis of the determined predictive temperature; and outputting the corrected blood glucose information. Therefore, even if the skin temperature rapidly rises or falls because of the external temperature, the electronic device can provide a result similar to the blood glucose value, measured by a disposable blood glucose meter, by correcting a blood glucose value in consideration of the temperature in the skin into which the sensor of a blood glucose meter for measuring blood glucose is inserted.

### TECHNICAL FIELD

The disclosure relates to an electronic device and a control method therefor, and more particularly, to an electronic device for providing blood glucose information based on a user's skin temperature and a control method therefor.

### BACKGROUND ART

In general, a blood glucose meter measures blood glucose through a sensor inserted into a user's skin, and corrects the measured blood glucose value based on a skin temperature sensed while the blood glucose is measured.

Particularly, the blood glucose meter makes a correction to output a value that is higher than the measured blood glucose value if the sensed skin temperature is low, and makes a correction to output a value that is lower than the measured blood glucose value if the sensed skin temperature is high.

The blood glucose meter correcting the blood glucose value based on the sensed skin temperature outputs a blood glucose value overcorrected from the measured blood glucose value, when the skin temperature is rapidly lowered or raised due to an external temperature.

As a result, when blood glucose is measured through the blood glucose meter in a state in which the skin temperature is rapidly lowered or raised due to an external temperature, there is a problem in that the blood glucose value is corrected by the blood glucose meter with a large margin error on the basis of a value measured by a disposable blood glucose meter.

### DISCLOSURE

### Technical Problem

An object of the disclosure is to measure user's blood glucose in consideration of an external temperature and a user's skin temperature.

### Technical Solution

According to an embodiment of the disclosure, an electronic device control method includes: receiving blood glucose information and skin temperature information from a blood glucose meter; obtaining external temperature information of a region in which a user wearing the blood glucose meter is located; determining, by using the skin temperature information and the external temperature information, a predictive temperature of an inner skin part at which an enzyme sensor of the blood glucose meter is located; correcting the blood glucose information on the basis of the determined predictive temperature; and outputting the corrected blood glucose information.

The determining may include: determining a predictive temperature of a first region of the inner skin part based on the skin temperature information and the external temperature information; determining a predictive temperature of a second region of the inner skin part based on preset deep part temperature information; and determining the predictive temperature of the inner skin part based on an average value of the predictive temperatures of the first and second regions.

In the determining of the predictive temperature of the first region, the predictive temperature of the first region may be determined based on the skin temperature information and a thermal diffusion table that is based on a length of the enzyme sensor of the blood glucose meter inserted into a skin.

In the determining of the predictive temperature of the first region, the skin temperature information may be compared with a predictive temperature that is calculated based on the external temperature information and a time of exposure to an external temperature, and if the two temperatures are different, the skin temperature information may be determined as the predictive temperature of the inner skin part.

In the determining of the predictive temperature of the second region, the predictive temperature of the second region may be determined based on the deep part temperature information, and a thermal diffusion table that is based on a distance from a skin surface to a point at which the deep part temperature information is measured and a length of the enzyme sensor.

The determining further may include determining a weight based on actual blood glucose information and the received blood glucose information. In the determining of the predictive temperature of the inner skin part, the predictive temperature of the inner skin part may be determined by applying the weight to the average value of the predictive temperatures of the first and second regions.

The determining of the weight may include: receiving actual blood glucose information measured under the same conditions as the received blood glucose information from an external device; determining an initial weight using the actual blood glucose information and the received blood glucose information; obtaining a temperature correction value corresponding to the initial weight with reference to a predefined temperature correction table; and determining the weight for temperature correction using the temperature correction value and the predictive temperature of the inner skin part.

The external temperature information may be sensed by at least one of an electronic device, a peripheral device that is communicable with the electronic device, or the blood glucose meter.

According to another embodiment of the disclosure, an electronic device includes: a communicator; an output unit; and

a processor configured to: receive blood glucose information and skin temperature information from a blood glucose meter through the communicator, obtain external temperature information of a region in which a user wearing the blood glucose meter is located, determine, by using the skin temperature information and the external temperature information, a predictive temperature of an inner skin part at which an enzyme sensor of the blood glucose meter is located, and correct the blood glucose information on the basis of the determined predictive temperature and control the output unit to output the corrected blood glucose information.

The processor may determine a predictive temperature of a first region of the inner skin part based on the skin temperature information and the external temperature information, determine a predictive temperature of a second region of the inner skin part based on preset deep part temperature information, and determine the predictive temperature of the inner skin part based on an average value of the predictive temperatures of the first and second regions.

The processor may determine the predictive temperature of the first region based on the skin temperature information and a thermal diffusion table that is based on a length of the enzyme sensor of the blood glucose meter inserted into a skin.

The processor may compare the skin temperature information with a predictive temperature that is calculated based on the external temperature information and a time of exposure to an external temperature, and if the two temperatures are different, determine the skin temperature information as the predictive temperature of the inner skin part.

The processor may determine the predictive temperature of the second region based on the deep part temperature information, and a thermal diffusion table that is based on a distance from a skin surface to a point at which the deep part temperature information is measured and a length of the enzyme sensor.

The processor may determine a weight based on actual blood glucose information and the received blood glucose information, and determine the predictive temperature of the inner skin part by applying the weight to the average value of the predictive temperatures of the first and second regions.

When receiving actual blood glucose information measured under the same conditions as the received blood glucose information from an external device, the processor may determine an initial weight using the actual blood glucose information and the blood glucose information, obtain a temperature correction value corresponding to the initial weight with reference to the predefined temperature correction table, and determines the weight for temperature correction using the temperature correction value and the predictive temperature of the inner skin part.

The external temperature information may be sensed by at least one of the electronic device, a peripheral device that is communicable with the electronic device, or the blood glucose meter.

According to another embodiment of the disclosure, there is provided a computer-readable recording medium coupled to an electronic device and having a program stored therein to execute the following steps: obtaining blood glucose information and skin temperature information from a blood glucose meter; obtaining external temperature information of a region in which a user wearing the blood glucose meter is located; determining, by using the skin temperature information and the external temperature information, a predictive temperature of an inner skin part at which an enzyme sensor of the blood glucose meter is located; correcting the blood glucose information on the basis of the determined predictive temperature; and outputting the corrected blood glucose information.

### Advantageous Effects

According to the various embodiments of the disclosure described above, even when the skin temperature is rapidly lowered or raised due to an external temperature, the electronic device is capable of correcting the blood glucose value in consideration of a temperature in a skin into which the sensor of the blood glucose meter for measuring blood glucose is inserted, thereby providing the result similar to the blood glucose value measured by the disposable blood glucose meter.

### DESCRIPTION OF DRAWINGS

FIG. 1A is a diagram of a blood glucose measurement system according to an embodiment of the disclosure.

FIG. 1B is a diagram of a blood glucose measurement system according to another embodiment of the disclosure.

FIG. 2 is a block diagram of a blood glucose meter according to an embodiment of the disclosure.

FIG. 3 is a schematic block diagram of an electronic device according to an embodiment of the disclosure.

FIG. 4 is an exemplary diagram illustrating a thermal diffusion table according to an embodiment of the disclosure.

FIG. 5 is an exemplary diagram illustrating a predictive temperature of an inner skin part according to an embodiment of the disclosure.

FIG. 6 is a detailed block diagram of the electronic device according to an embodiment of the disclosure.

FIG. 7 is an exemplary diagram illustrating a blood glucose profile generated based on the general blood glucose measurement.

FIG. 8 is an exemplary diagram illustrating a blood glucose profile generated using blood glucose information corrected based on the predictive temperature of the inner skin part in the electronic device according to an embodiment of the disclosure.

FIG. 9 is a flowchart of a method for correcting blood glucose information in the electronic device according to an embodiment of the disclosure.

FIG. 10 is a flowchart of a method for determining a predictive temperature of an inner skin part into which an enzyme sensor of the blood glucose meter is inserted in the electronic device according to an embodiment of the disclosure.

FIG. 11 is a flowchart of a method for setting a weight used to determine a predictive temperature of an inner skin part into which the enzyme sensor of the blood glucose meter is inserted in the electronic device according to an embodiment of the disclosure.

### BEST MODE

Since the disclosure may be variously modified and have several embodiments, specific embodiments will be illustrated in the drawings and described in detail in the detailed description. However, it is to be understood that the disclosure is not limited to specific embodiments, but includes all modifications, equivalents, and substitutions included in the spirit and the scope of the disclosure. In describing the embodiments of the disclosure, when it is determined that a detailed description of the known art related to the disclosure may obscure the gist of the disclosure, the detailed description thereof will be omitted.

The terms “first”, “second”, and the like, may be used to describe various components, but the components are not to be construed as being limited by these terms. The terms are used only to distinguish one component from another component.

Terms used herein are used only to describe specific embodiments, and are not intended to limit the scope. Singular forms include plural forms unless the context clearly indicates otherwise. It should be further understood that term “include”, “formed of”, or the like used herein specifies the presence of features, numerals, steps, operations, components, parts, or combinations thereof described in the specification, but does not preclude the presence or addition of one or more other features, numerals, steps, operations, components, parts, or combinations thereof.

In the embodiments, a ‘module’ or ‘unit’ performs at least one function or operation, and may be implemented by hardware, software, or a combination of hardware and software. In addition, a plurality of ‘modules’ or a plurality of ‘units’ are integrated into at least one module, except for the ‘module’ or ‘unit’ which needs to be implemented by particular hardware, and thus may be implemented by at least one processor (not shown).

Hereinafter, embodiments of the disclosure will be described in detail with reference to the accompanying drawings. The same reference numerals will be used to describe the same or corresponding components, and overlapping descriptions thereof will be omitted.

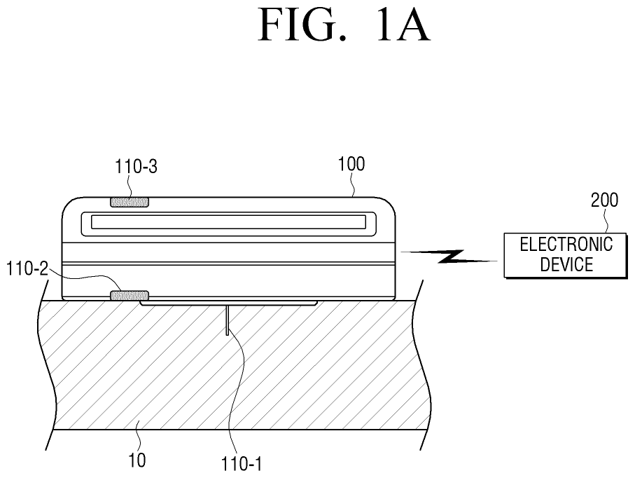

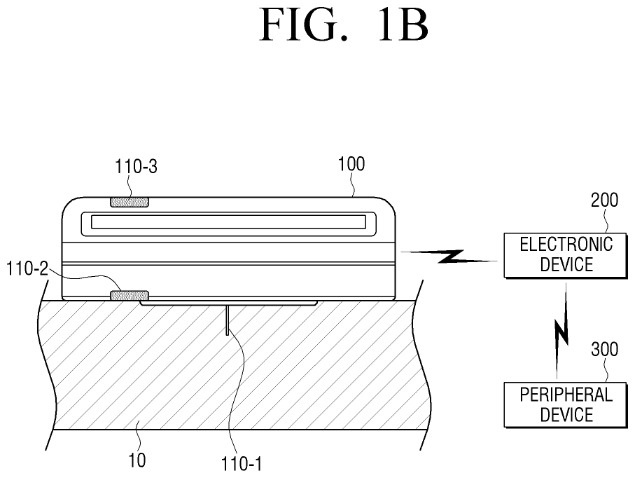

FIG. 1A is a diagram of a blood glucose measurement system according to an embodiment of the disclosure, and FIG. 1B is a diagram of a blood glucose measurement system according to another embodiment of the disclosure.

As illustrated in FIG. 1A, a blood glucose measurement system includes a blood glucose meter 100 and an electronic device 200. The blood glucose meter 100 is a device attached to a user's body to measure user's blood glucose.

The electronic device 200 may be a device that performs wireless short-range communication with the blood glucose meter 100 and provides a user's blood glucose condition based on blood glucose information measured by the blood glucose meter 100. Furthermore, the electronic device 200 may provide blood glucose management information based on the received blood glucose information. The electronic device 200 may be a display device, such as a smart phone, or a wearable device, such as a smart watch, a smart band, or a smart glass (AR).

Specifically, the blood glucose meter 100 includes an enzyme sensor 110-1 and a temperature sensor 110-2. The enzyme sensor 110-1 is a sensor inserted into a user's skin to measure user's blood glucose, and the skin temperature sensor 110-2 is a sensor provided at one side surface of the blood glucose meter 100 that is in contact with the user's skin to sense a user's skin temperature.

Accordingly, the blood glucose meter 100 determines a current value corresponding to a blood glucose value measured by the enzyme sensor 110-1 inserted into the user's skin. In addition, the blood glucose meter 100 obtains skin temperature information sensed by the temperature sensor 110-2 at the time when the blood glucose value is measured by the enzyme sensor 110-1. Thereafter, the blood glucose meter 100 transmits to the electronic device 200 the blood glucose information including the current value corresponding to the measured blood glucose value and the skin temperature information sensed at the time when the blood glucose value is measured.

When the blood glucose information and the skin temperature information are received from the blood glucose meter 100, the electronic device 200 obtains external temperature information. Here, the external temperature information may be a temperature measured in a region in which a user wearing the blood glucose meter 100 is located. Meanwhile, the electronic device 200 may obtain the external temperature information through the embodiments that will be described below.

According to an embodiment, the electronic device 200 may obtain a temperature value sensed by a temperature sensor (not shown) included in the electronic device 200 as external temperature information of the region in which the user wearing the blood glucose meter 100 is located.

According to another embodiment, the electronic device 200 may receive and obtain external temperature information from the blood glucose meter 100. As illustrated in FIG. 1A, the blood glucose meter 100 may further include an external temperature sensor 110-3 for sensing an external temperature, in addition to the skin temperature sensor 110-2 for sensing a user's skin temperature. In this case, the blood glucose meter 100 may transmit to the electronic device 200 the blood glucose information, the skin temperature information, and the external temperature information sensed by the external temperature sensor 110-3. Accordingly, the electronic device 200 may obtain external temperature information from the blood glucose meter 100.

According to another embodiment, the electronic device 200 may receive and obtain external temperature information sensed by a peripheral device 300 from the peripheral device 300 that is communicable with the electronic device 200, as illustrated in FIG. 1B. Here, the peripheral device 300 may be a device that is capable of sensing an external temperature, for example a smart air conditioner, a smart air cleaner, or a smart phone.

Specifically, when the blood glucose information and the skin temperature information are received from the blood glucose meter 100, the electronic device 200 transmits a signal for requesting external temperature information to the communicable peripheral device 300. Accordingly, the peripheral device 300 may sense an external temperature and transmit the sensed external temperature information to the electronic device 200.

Therefore, the electronic device 200 may obtain external temperature information from the peripheral device 300.

Once the external temperature information is obtained through the various embodiments, the electronic device 200 determines a predictive temperature of an inner skin part at which the enzyme sensor 110-1 of the blood glucose meter 100 is located, using the skin temperature information received from the blood glucose meter 100 and the obtained external temperature information. Thereafter, the electronic device 200 corrects the blood glucose information received from the blood glucose meter 100 based on the predictive temperature of the inner skin part, and outputs the corrected blood glucose.

However, the disclosure is not limited thereto, and the blood glucose meter 100 may determine a predictive temperature of an inner skin part at which the enzyme sensor 110-1 is located, using the skin temperature information and the external temperature information sensed through the skin temperature sensor 110-2 and the external temperature sensor 110-3. Thereafter, the blood glucose meter 100 may correct the blood glucose value measured by the enzyme sensor 110-1 based on the predictive temperature of the inner skin part, and transmit blood glucose information including a current value corresponding to the corrected blood glucose value to the electronic device 200.

Also, the disclosure is not limited thereto. When the blood glucose information and the skin temperature information are received from the blood glucose meter 100, the electronic device 200 transmits the received skin temperature information and the obtained external temperature information to an external server (not shown). Accordingly, the external server (not shown) determines a predictive temperature of an inner skin part at which the enzyme sensor 110-1 of the blood glucose meter 100 is located using the skin temperature information and the external temperature information received from the electronic device 200, and transmits the determined predictive temperature to the electronic device 200. Therefore, the electronic device 200 may correct the blood glucose information received from the blood glucose meter 100 based on the predictive temperature received from the external server (not shown), and output the corrected blood glucose information.

In the disclosure, the operations of the electronic device 200 will be described in detail as to how to determine a predictive temperature of an inner skin part at which the enzyme sensor 100-1 of the blood glucose meter 100 is located, and correct the blood glucose information based on the determined predictive temperature.

The blood glucose measurement system according to the disclosure has been briefly described so far. Hereinafter, the blood glucose meter 100 and the electronic device 200 according to the disclosure will be described in detail.

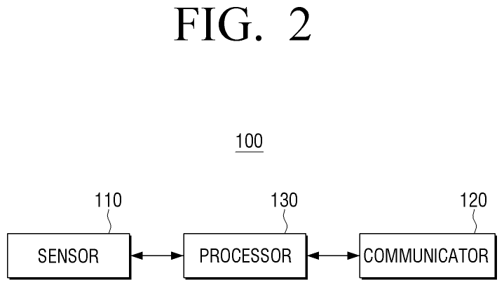

FIG. 2 is a block diagram of the blood glucose meter according to an embodiment of the disclosure.

As illustrated in FIG. 2, the blood glucose meter 100 includes a sensor 110, a communicator 120, and a processor 130.

The sensor 110 includes an enzyme sensor 110-1 and a skin temperature sensor 110-2. The enzyme sensor 110-1 is a sensor for measuring user's blood glucose, and may be implemented in a needle type so as to be inserted into a user's skin. The skin temperature sensor 110-2 is provided within one side surface of the blood glucose meter 100 that is in contact with the user's skin to sense a user's skin temperature. Additionally, the sensor 110 may further include an external temperature sensor 110-3 for sensing an external temperature, as described above.

The communicator 120 performs wireless data communication with the electronic device 200. According to an embodiment, the communicator 120 may include a short-range communication module such as Bluetooth or Zigbee, and may perform wireless data communication with the electronic device 200 through the short-range communication module.

The processor 130 controls overall operations of individual components constituting the blood glucose meter 100. In particular, the processor 130 controls the enzyme sensor 110-1 to periodically measure user's blood glucose. Furthermore, the processor 130 controls the skin temperature sensor 110-2 to periodically sense a user's skin temperature. That is, the processor 130 controls the skin temperature sensor 110-2 to sense a user's skin temperature at the time when blood glucose is measured through the enzyme sensor 110-1.

Following such a control command, the enzyme sensor 110-1 periodically may measure user's blood glucose, and the skin temperature sensor 110-2 may sense a user's skin temperature at the time when the user's blood glucose is measured through the enzyme sensor 110-1.

Meanwhile, the processor 130 determines a current value corresponding to the blood glucose value measured through the enzyme sensor 110-1, and controls the communicator 120 to transmit to the electronic device 200 the blood glucose information including the determined current value and the skin temperature information sensed by the skin temperature sensor 110-2. Accordingly, the communicator 120 may transmit the user's blood glucose information and skin temperature information to the electronic device 200.

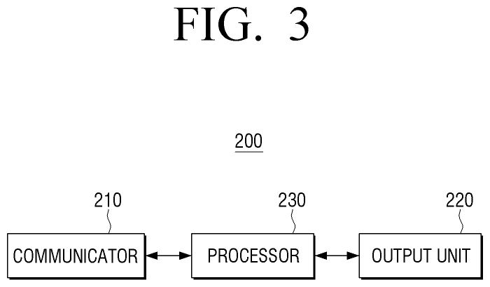

FIG. 3 is a schematic block diagram of an electronic device according to an embodiment of the disclosure.

As illustrated in FIG. 3, the electronic device 200 includes a communicator 210, an output unit 220, and a processor 230.

The communicator 210 performs wireless data communication with the blood glucose meter 100. Specifically, the communicator 210 performs data communication with the blood glucose meter 100, and receives the user's blood glucose information and the user's skin temperature information from the blood glucose meter 100. In a case where the electronic device 200 is not capable of sensing an external temperature, the communicator 210 may receive external temperature information from a peripheral device 300 within a region in which a user wearing the blood glucose meter 100 is located, among a plurality of peripheral devices 300 that are capable of sensing an external temperature.

The output unit 220 outputs user's blood glucose information corrected based on the user's skin temperature and the ambient temperature, the blood glucose management information based on the blood glucose information, and the like as at least one of an image or an audio.

The processor 230 controls overall operations of individual components constituting the electronic device 200. In particular, when the blood glucose information and the skin temperature information are received from the blood glucose meter 100, the processor 230 obtains external temperature information of a region in which a user wearing the blood glucose meter 100 is located.

Specifically, the electronic device 200 may include a temperature sensor for sensing an external temperature. In this case, the processor 230 may obtain from a sensor 270 external temperature information of a region in which a user wearing the blood glucose meter 100 is located.

Meanwhile, in a case where the electronic device 200 is not capable of sensing an external temperature, the processor 230 controls the communicator 210 to receive external temperature information from a peripheral device 300 within a region in which a user wearing the blood glucose meter 100 is located, among a plurality of peripheral devices 300 that are capable of sensing an external temperature. Accordingly, the communicator 210 may request and receive external temperature information to/from the peripheral device 300 within the region in which the user wearing the blood glucose meter 100 is located. Therefore, the processor 230 may obtain the external temperature information received from the peripheral device 300 through the communicator 210.

Thereafter, the processor 230 determines a predictive temperature of an inner skin part at which the enzyme sensor 110-1 of the blood glucose meter 100 is located, using the skin temperature information received from the blood glucose meter 100 and the external temperature information. Subsequently, the processor 230 corrects the blood glucose information received from the blood glucose meter 100 based on the determined predictive temperature of the inner skin part, and controls the output unit 220 to output the corrected blood glucose information. Accordingly, the output unit 220 may output the blood glucose information corrected based on the predictive temperature of the inner skin part as at least one of an image or an audio.

Specifically, the processor 230 determines a predictive temperature of a first region of the inner skin part based on the skin temperature information received from the blood glucose meter 100 and the obtained external temperature information.

According to an embodiment, the processor 230 may determine the predictive temperature of the inner skin part based on the skin temperature information received from the blood glucose meter 100 and a heat diffusion table.

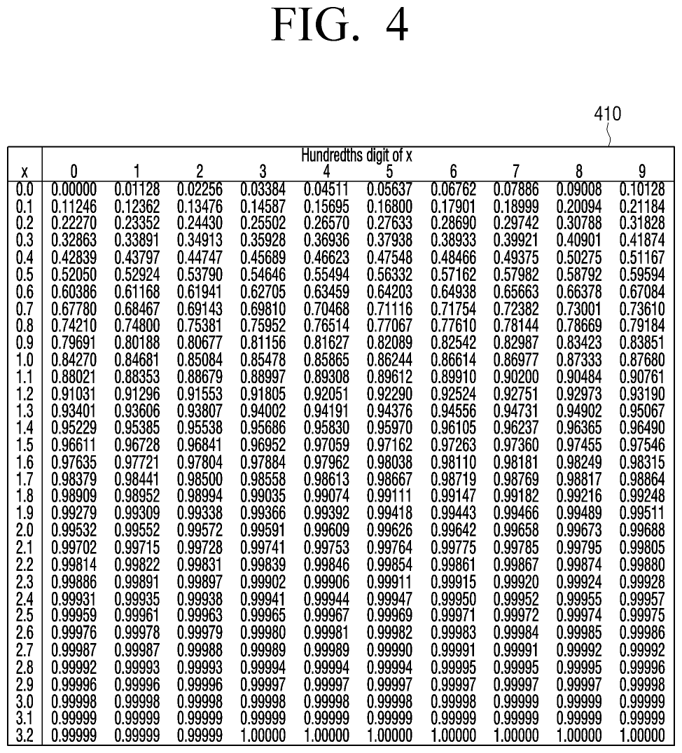

FIG. 4 is an exemplary diagram illustrating a thermal diffusion table according to an embodiment of the disclosure.

As illustrated in FIG. 4, the heat diffusion table 410 is a table that defines a heat diffusion value at which heat particles are transferred into a skin, depending on a length of the enzyme sensor 110-1 of the blood glucose meter 100.

Therefore, the processor 230 may determine the predictive temperature of the first region of the inner skin part based on the skin temperature information received from the blood glucose meter 100 and the heat diffusion table that is based on the length of the enzyme sensor 110-1 of the blood glucose meter 100 inserted into the skin.

Meanwhile, the processor 230 calculates a predictive temperature based on the obtained external temperature information and a time exposed to an external temperature (hereinafter referred to as a predictive temperature of an outer skin part), and compares the calculated predictive temperature of the outer skin part and the skin temperature information received from the blood glucose meter 100. If the two temperatures are different as a result of comparison, the processor 230 may determine the skin temperature information received from the blood glucose meter 100 as the predictive temperature of the first region of the inner skin part.

However, the disclosure is not limited thereto. If the difference between the skin temperature (T₁) and the predictive temperature (Predicted T₁) of the outer skin part is not within a preset threshold range, the processor 230 may determine the skin temperature information received from the blood glucose meter 100 as the predictive temperature of the first region of the inner skin part.

Meanwhile, the predictive temperature of the first region may be calculated based on the following Equation 1, and the predictive temperature based on the external temperature information and the time of exposure to the external temperature may be calculated based on the following Equation 2.

$${T}_{A} ( x , t ) = {T}_{1} [ 1 - \text{erf} ( \frac{x}{2 \sqrt{Dt}} ) ] \qquad [ Equation 1 ]$$

Here, T_{A}(x,t) is a predictive temperature of the first region, T₁ is a skin temperature included in the skin temperature information received from the blood glucose meter 100, and x is a length of the enzyme sensor 110-1. Further, D is a thermal diffusion coefficient, and t may be a time during which the skin temperature is changed.

Predicted T₁=T₂+f(T₂)t [Equation 2]

Here, Predicted T₁ is a predictive temperature of the outer skin part, T₂ is an external temperature included in the external temperature information, t may be a time during which the skin is exposed to the external temperature. Meanwhile, if t≤600 sec, f(T₂) may be 5E⁻⁵T−0.0015, and if t>600 sec, f(T₂) may be 0.0002T₂−0.0072.

When the skin temperature (T₁) included in the skin temperature information received from the blood glucose meter 100 is compared with the predictive temperature (Predicted T₁) of the outer skin part, if the two temperatures are different, the skin temperature information received from the blood glucose meter 100 may be determined as the predictive temperature (T_{A}(x,t)) of the first region of the inner skin part.

Meanwhile, if the skin temperature (T₁) and the predictive temperature (Predicted T₁) of the outer skin part are identical, the processor 230 may determine a value calculated based on the aforementioned Equation 1 as the predictive temperature of the first region of the inner skin part.

However, the disclosure is not limited thereto. If the difference between the skin temperature (T₁) and the predictive temperature (Predicted T₁) of the outer skin part is in the preset threshold range, the value calculated based on the aforementioned Equation 1 may be determined as the predictive temperature of the first region of the inner skin part.

The processor 230 determines a predictive temperature of a second region of the inner skin part based on preset deep part temperature information. Specifically, the processor 230 may determine the predictive temperature of the second region of the inner skin part based on the deep part temperature information, and a thermal diffusion table that is based on a distance from a skin surface to a point at which the deep part temperature information is measured and a length of the enzyme sensor 110-1. Here, the deep part temperature information may be a standard temperature of a body, for example 36.5° C.

The predictive temperature of the second region may be calculated based on the following Equation 3.

$${T}_{B} ( x , t ) = {T}_{d} [ 1 + \text{erf} ( \frac{5 - x}{2 \sqrt{Dt}} ) ] \qquad [ Equation 3 ]$$

Here, T_{B}(x,t) is a predictive temperature of the second region of the inner skin part, and T_{d} may be deep part temperature information. Further, x is a length of the enzyme sensor 110-1, D is a thermal diffusion coefficient, and t may be a time during which the skin temperature is changed.

Once the predictive temperatures of the first and second regions of the inner skin part are determined through the aforementioned Equations 1 to 3, the processor 230 may determine an average value of the predictive temperatures of the first and second regions as the predictive temperature of the inner skin part. Thereafter, the processor 230 may correct the blood glucose information received from the blood glucose meter 100 based on the predictive temperature of the inner skin part, and output the corrected blood glucose information through the output unit 220. Specifically, the processor 230 determines a blood glucose value based on the current value included in the blood glucose information received from the blood glucose meter 100. Subsequently, the processor 230 may correct the determined blood glucose value based on the predictive temperature of the inner skin part, and output the corrected blood glucose information through the output unit 220.

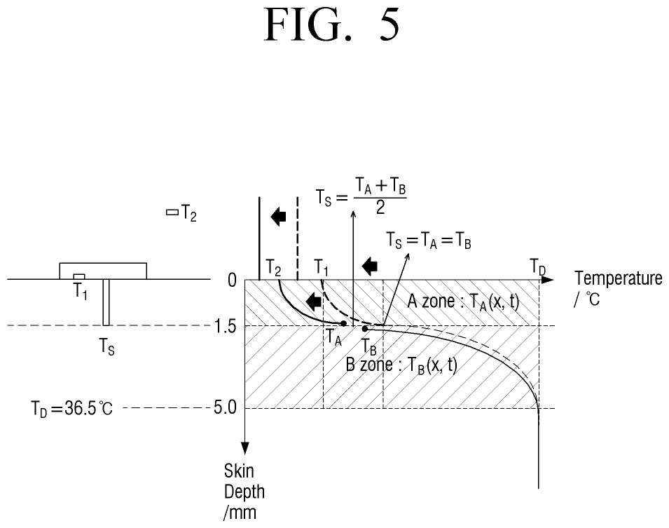

FIG. 5 is an exemplary diagram illustrating a predictive temperature of an inner skin part according to an embodiment of the disclosure.

As illustrated in FIG. 5, the first region (A zone) may be a region in which the enzyme sensor 110-1 is inserted into a skin, and the second region (B zone) may be a region from a boundary point of the first region to a point at which the deep part temperature information is measured.

The temperature of the first region (A zone) of the inner skin part may vary depending on an external temperature, and the temperature of the second region (B zone) of the inner skin part may be maintained at a certain level regardless of the external temperature.

If the temperature of the first region (A zone) of the inner skin part is changed according to the external temperature, the predictive temperature (Ts) of the inner skin part may be determined as an average value of the predictive temperatures of the first and second regions.

On the other hand, if the temperature of the first region (A zone) of the inner skin part is not changed according to the external temperature, the predictive temperature (Ts) of the inner skin part, the predictive temperature of the first region and the predictive temperature of the second region may be determined as the same value.

According to a further aspect of the disclosure, the processor 230 may determine the predictive temperature of the inner skin part by applying a preset weight to the average value of the predictive temperatures of the first and second regions. Specifically, the processor 230 determines a weight to be applied to the predictive temperature of the inner skin part, based on actual blood glucose information measured by a disposable blood glucose meter (not shown) and the blood glucose information received from the blood glucose meter 100 as described above.

More specifically, the electronic device 200 may receive the actual blood glucose information measured by the disposable blood glucose meter (not shown) through the communicator 210. Here, the actual blood glucose information may be information measured under the same conditions as the blood glucose information received from the blood glucose meter 100. At this point, the same conditions may be a time and a place at which blood glucose is measured by the disposable blood glucose meter (not shown) and by the blood glucose meter 100.

Once the actual blood glucose information is received, the processor 230 determines an initial weight using the received actual blood glucose information and the blood glucose information received from the blood glucose meter 100. That is, the processor 230 may determine a value obtained by dividing a blood glucose value (Yc) included in the actual blood glucose information by a blood glucose value (Xc) included in the received blood glucose information as the initial weight (Wc).

Thereafter, the processor 230 obtains a temperature correction value (Tc) corresponding to the initial weight (Wc), with reference to a predefined temperature correction table. Subsequently, the processor 230 determines a weight for temperature correction using the temperature correction value (Tc) and the predictive temperature (Ts) of the inner skin part that is used to obtain the corrected blood glucose information. Specifically, the processor 230 may determine a value obtained by dividing the temperature correction value (Tc) by the predictive temperature (Ts) of the inner skin part as the weight (β) for temperature correction.

Thereafter, the processor 230 may finally determine the predictive temperature of the inner skin part by multiplying the average value of the predictive temperatures of the first and second regions of the inner skin part calculated through the aforementioned Equations 1 to 3 by the weight (β) for temperature correction. Subsequently, the processor 230 may correct the blood glucose information received from the blood glucose meter 100 based on the predictive temperature of the inner skin part, and output the corrected blood glucose information.

Hereinafter, the detailed configuration of the above-described electronic device 200 will be specifically described.

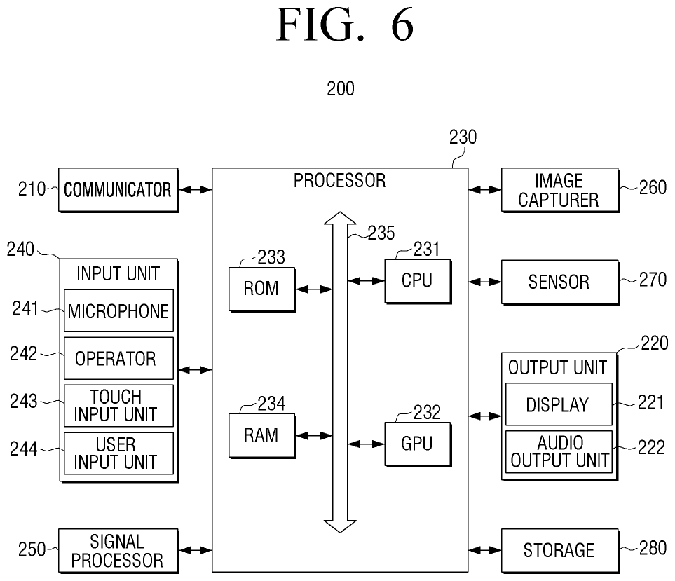

FIG. 6 is a detailed block diagram of the electronic device according to an embodiment of the disclosure.

As illustrated in FIG. 6, the electronic device 200 may include a communicator 210, an output unit 220, a processor 230, an input unit 240, a signal processor 250, an image capturer 260, a sensor 270, and a storage 280.

As described above, the communicator 210 may perform data communication with the blood glucose meter 100, and receive blood glucose information and skin temperature information measured by the blood glucose meter 100. In addition, in a case where the electronic device 200 is not capable of measuring an external temperature, the communicator 210 may receive external temperature information from a peripheral device 300 within a region in which a user wearing the blood glucose meter 100 is located.

The communicator 210 may include a short-range communication module (not shown) for performing wireless short-range communication with the blood glucose meter 100 and the peripheral device 300. According to an embodiment, the short-range communication module (not shown) may include at least one of a Bluetooth module, an infrared data association (IrDA) module, a near field communication (NFC) module, a wireless fidelity (WIFI) module, or a Zigbee module.

In addition, the communicator 210 may further include a wireless communication module (not shown) for performing wireless data communication with a web server (not shown), a content server (not shown), or the like. Here, the wireless communication module (not shown) may be a module connected to an external network to perform communication according to a wireless communication protocol such as an Institute of Electrical and Electronics Engineers (IEEE), or a mobile communication module accessing a mobile communication network to perform communication according to various mobile communication standards such as 3rd Generation (3G), 3rd Generation Partnership Project (3GPP), and Long Term Evolution (LTE).

The communicator 210 may be implemented by the various wireless communication schemes described above, and other communication techniques that are not mentioned herein may be employed as required.

In addition, the communicator 210 may further include a connector (not shown) including at least one wired communication module such as a High-Definition Multimedia Interface (HDMI), a Universal Serial Bus (USB), and an Institute of Electrical and Electronics Engineers (IEEE) 1394. The connector (not shown) may receive content data transmitted from an external server (not shown) via a wired cable connected to the connector (not shown) according to a control command of the processor 230, or transmit the stored content data to an external recording medium. In addition, the connector (not shown) may receive power from a power source via the wired cable that is physically connected to the connector (not shown).

The output unit 220 described above may include a display 221 and an audio output unit 222. The display 221 may display on a screen not only an image for the corrected blood glucose information but also an execution user interface (UI) of an application or an image of content requested by a user. The audio output unit 222 may output an audio signal for the corrected blood glucose information, an audio signal of the content, or the like in an audible manner through a speaker.

Meanwhile, the display 221 may be implemented as a Liquid Crystal Display (LCD), an Organic Light Emitting Display (OLED), or the like. In particular, the display 221 may be implemented in the form of a touch screen that forms a mutual layer structure together with a touch input unit 243 included in the input unit 240, which will be described below.

The processor 230 controls overall operations of individual component constituting the electronic device 200. In particular, as described above, the processor 230 determines a predictive temperature of the inner skin part using the skin temperature information received from the blood glucose meter 100 and the obtained external temperature information, and corrects the blood glucose information received from the blood glucose meter 100 based on the determined predictive temperature. The particular operations of the processor 230 have been described in detail above, and therefore the detailed description thereof will be omitted hereinbelow.

The input unit 240, which is an input means for receiving various user commands to be transmitted to a processor 230, includes a microphone 241, an operator 242, a touch input unit 243, and a user input unit 244.

The microphone 241 receives a user's voice command, and the operator 242 may be implemented as a key pad including various function keys, number keys, special keys, character keys, and the like. When the display 221 described above is implemented in the form of a touch screen, the touch input unit 243 may be implemented as a touch pad forming a mutual layer structure with the display 221. In this case, the touch input unit 243 may receive a selection command for at least one of various application-related icons and execution UIs for running applications which are displayed through the display 221.

The user input unit 244 may receive an infrared ray (IR) signal or a radio frequency (RF) signal for controlling the operation of the electronic device 200 from a remote controller and a control device (not shown).

The signal processor 250 may be a component for processing image data and audio data of content received through the communicator 210 or content stored in the storage 280, which will be described below, according to a control command of the processor 230. Specifically, the signal processor 250 may perform various image processing, such as decoding, scaling, noise filtering, frame rate conversion, and resolution conversion, for the image data included in the content. Furthermore, the signal processor 250 performs various audio signal processing, such as decoding, amplification, noise filtering, and the like, for the audio data included in the content.

The image capturer 260, which is for capturing a still image or a moving image according to a user command, may be implemented in a plural number, including, for example, a front camera and a rear camera.

The sensor 270 is a sensor that senses an ambient brightness, an external temperature, and a motion of the electronic device 200. The sensor 270 may include an illumination sensor (not shown), a temperature sensor (not shown), an accelerometer sensor (not shown), a magnetic sensor (not shown), a gravity sensor (not shown), a gyroscope sensor (not shown), and the like.

The illumination sensor (not shown) may be a sensor that senses a brightness of an ambient environment, and the temperature sensor (not shown) may be a sensor that senses an external temperature.

The accelerometer sensor (not shown) is an accelerometer sensor that measures an acceleration or an impact strength of the electronic device 200 that is moving. Further, the magnetic sensor (not shown) is a sensor that is capable of detecting an azimuth angle using the Earth's magnetic field, and the gravity sensor (not shown), which is a sensor that detects which direction the gravity acts in, automatically rotates along a direction in which a user holds the electronic device 200 so as to sense a direction. Lastly, the gyroscope sensor (not shown) is a sensor that helps to recognize a more detailed and precise motion by adding rotations to the existing motion sensor (not shown) to recognize the motion in six-axis directions.

The storage 280 stores various kinds of information for correcting the blood glucose information received from the blood glucose meter 100. In addition, the storage 280 may store programs for executing various applications, contents, and various operation programs for controlling the operations of the electronic device 200. Here, the operating programs may be programs that are read and compiled in the storage 280 to operate individual components of the electronic device 200, when the electronic device 200 is turned on.

Meanwhile, the processor 230 described above may include a central processing unit (CPU) 231, a graphic processing unit (GPU) 232, a read only memory (ROM) 233, and a random access memory (RAM) 234, and the CPU 231, the GPU 232, the ROM 233, and the RAM 234 may be connected to each other via a bus 235.

The CPU 231 accesses the storage 280, and performs booting using an operating system (OS) stored in the storage 280. The CPU 231 also performs various operations using various programs, contents, data, and the like stored in the storage 280.

The GPU 232 generates a display screen including various objects such as icons, images and texts. Specifically, the GPU 232 calculates attribute values such as a coordinate value, a shape, a size and a color in which each object is to be displayed according to the layout of the screen based on the received control command, and generates display screens in various layouts including objects based on the calculated attribute values.

The ROM 233 has a command set for booting a system stored therein. When a turn-on command is input and power is supplied, the CPU 231 copies the OS stored in the storage 280 to the RAM 234 according to the command stored in the ROM 233, and executes the OS to boot the system. Once the booting is completed, the CPU 231 copies various programs stored in the storage 280 to the RAM 234, and executes the programs copied to the RAM 234 to perform various operations.

The processor 230 may be combined with each of the components described above and implemented as a system-on-a-chip (SoC).

Meanwhile, the operations of the processor 230 described above may be performed by the programs stored in the aforementioned storage 280. Here, the storage 280 may be implemented as at least one of a memory card (e.g. an SD card, a memory stick), a nonvolatile memory, a volatile memory, or a hard disk drive (HDD) , or a solid state drive (SSD) that is attachable or detachable to/from the ROM 233, the RAM 234, or the electronic device 200.

The operations of the individual components constituting the electronic device 200 according to the disclosure have been described in detail so far.

Hereinafter, the results about the blood glucose information before and after applying the predictive temperature of the inner skin part determined by the electronic device 200 according to the disclosure will be described.

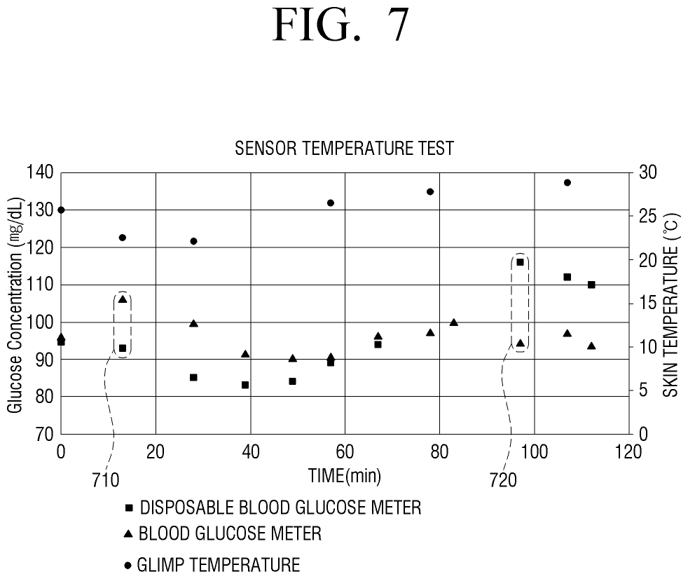

FIG. 7 is an exemplary diagram illustrating a blood glucose profile generated based on the general blood glucose measurement.

As illustrated in FIG. 7, the blood glucose profile may include actual blood glucose information measured periodically through a disposable blood glucose meter (disposable blood glucose measurement device) and blood glucose information corrected based on the skin temperature from the blood glucose meter 100 (continuous blood glucose meter) at the same time when the actual blood glucose information is measured.

Specifically, the blood glucose meter 100 measures blood glucose at the time when actual blood glucose information is measured through the disposable blood glucose meter (not shown). Also, the blood glucose meter (100) senses a skin temperature at the time when the blood glucose value is measured. Thereafter, the blood glucose meter 100 corrects the measured blood glucose value based on the sensed skin temperature.

Meanwhile, the skin temperature may be a temperature of an outer skin part, and the temperature of the outer skin part directly exposed to an external temperature causes a temperature of the inner skin part to be changed according to the change in the external temperature. That is, when the external temperature is high, the temperature of the outer skin part is measured as being relatively higher than that of the inner skin part, and when the external temperature is low, the temperature of the outer skin part is measured as being relatively lower than that of the inner skin part.

For example, the disposable blood glucose meter (not shown) may measure a blood glucose value between 90 and 100 mg/dL at a first point 710. Meanwhile, the temperature of the outer skin part may be sensed as being low due to an external temperature of a region in which a user wearing the blood glucose meter 100 is located at the first point 710. In this case, the blood glucose meter 100 overcorrects the blood glucose value based on the sensed temperature of the outer skin part so that the blood glucose value is measured to be higher than the blood glucose value measured through the enzyme sensor 110-1. As a result, the blood glucose meter 100 may determine a blood glucose value corrected to be between 100 and 110 mg/dL as user's blood glucose information at the first point 710.

Therefore, the blood glucose value determined as the user blood glucose information through the blood glucose meter 100 at the first point 710 has an error of about +10 mg/dL on the basis of the blood glucose value measured through the disposable blood glucose meter (not shown).

Meanwhile, the disposable blood glucose meter (not shown) may measure a blood glucose value between 110 and 120 mg/dL at a second point 720. Further, the temperature of the outer skin part may be sensed as being high due to an external temperature of a region in which a user wearing the blood glucose meter 100 is located at the second point 720. In this case, the blood glucose meter 100 overcorrects the blood glucose value based on the sensed temperature of the outer skin part so that the blood glucose value is measured to be lower than the blood glucose value measured through the enzyme sensor 110-1. As a result, the blood glucose meter 100 may determine a blood glucose value corrected to be between 90 and 100 mg/dL as user's blood glucose information at the second point 720.

Therefore, the blood glucose value determined as the user blood glucose information through the blood glucose meter 100 at the second point 720 has an error of about −20 mg/dL on the basis of the blood glucose value measured through the disposable blood glucose meter (not shown).

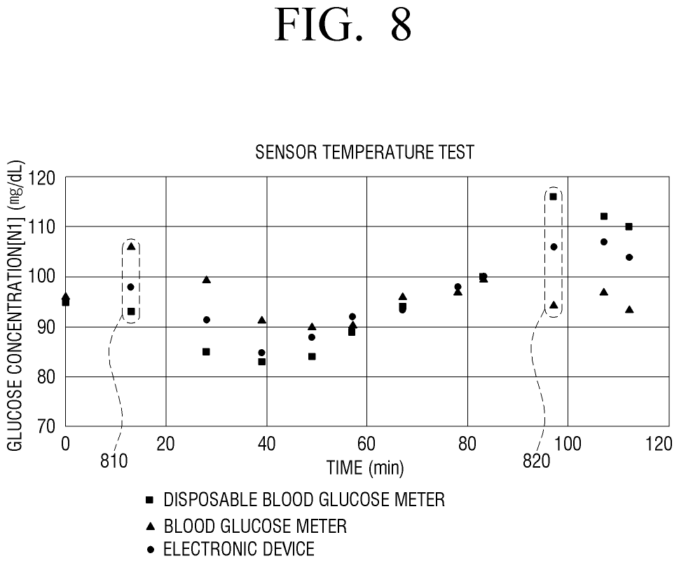

FIG. 8 is an exemplary diagram illustrating a blood glucose profile generated using blood glucose information corrected based on the predictive temperature of the inner skin part in the electronic device according to an embodiment of the disclosure.

As illustrated in FIG. 8, the blood glucose profile may include actual blood glucose information measured periodically through the disposable blood glucose meter (disposable blood glucose measurement device) and blood glucose information corrected based on the skin temperature from the blood glucose meter 100 (continuous blood glucose meter) at the same time when the actual blood glucose information is measured. In addition, the blood glucose profile may include blood glucose information corrected based on the predictive temperature of the inner skin part that is determined by the electronic device 200 according to the disclosure based on the external temperature and the skin temperature.

For example, the disposable blood glucose meter (not shown) may measure a blood glucose value between 90 and 100 mg/dL at a first point 810. Meanwhile, the temperature of the outer skin part may be sensed as being low due to an external temperature of a region in which a user wearing the blood glucose meter 100 is located at the first point 810. In this case, the blood glucose meter 100 overcorrects the blood glucose value based on the sensed temperature of the outer skin part so that the blood glucose value is measured to be higher than the blood glucose value measured through the enzyme sensor 110-1. As a result, the blood glucose meter 100 may determine a blood glucose value corrected to be between 100 and 110 mg/dL as user's blood glucose information at the first point 810.

Therefore, the blood glucose value determined as the user blood glucose information through the blood glucose meter 100 at the first point 810 has an error of about +10 mg/dL, on the basis of the blood glucose value measured through the disposable blood glucose meter (not shown).

Meanwhile, the electronic device 200 according to the disclosure determines a predictive temperature of an inner skin part into which the enzyme sensor 110-1 of the blood glucose meter 100 is inserted, based on the external temperature sensed at the first point 810 and the skin temperature sensed by the blood glucose meter 100. Thereafter, the electronic device 200 corrects the blood glucose information received from the blood glucose meter 100 based on the predictive temperature of the inner skin part. As a result, the electronic device 200 may determine a blood glucose value corrected to be between 95 and 100 mg/dL as user's blood glucose information at the first point 810.

Therefore, the electronic device 200 according to the disclosure may correct the blood glucose value to have an error range smaller than the blood glucose value corrected by the blood glucose meter 100, on the basis of the blood glucose value measured through the disposable blood glucose meter (not shown).

Meanwhile, the disposable blood glucose meter (not shown) may measure a blood glucose value between 110 and 120 mg/dL at a second point 820. Further, the temperature of the outer skin part may be sensed as being high due to an external temperature of a region in which a user wearing the blood glucose meter 100 is located at the second point 820. In this case, the blood glucose meter 100 overcorrects the blood glucose value based on the sensed temperature of the outer skin part so that the blood glucose value is measured to be lower than the blood glucose value measured through the enzyme sensor 110-1. As a result, the blood glucose meter 100 may determine a blood glucose value corrected to be between 90 and 100 mg/dL as user's blood glucose information at the second point 820.

Therefore, the blood glucose value determined as the user blood glucose information through the blood glucose meter 100 at the second point 820 has an error of about −20 mg/dL, on the basis of the blood glucose value measured through the disposable blood glucose meter (not shown).

Meanwhile, the electronic device 200 according to the disclosure determines a predictive temperature of an inner skin part into which the enzyme sensor 110-1 of the blood glucose meter 100 is inserted, based on the external temperature sensed at the second point 820 and the skin temperature sensed by the blood glucose meter 100. Thereafter, the electronic device 200 corrects the blood glucose information received from the blood glucose meter 100 based on the predictive temperature of the inner skin part. As a result, the electronic device 200 may determine a blood glucose value corrected to be between 100 and 110 mg/dL as user's blood glucose information at the second point 820.

Therefore, the electronic device 200 according to the disclosure may correct the blood glucose value to have an error range smaller than the blood glucose value corrected by the blood glucose meter 100, on the basis of the blood glucose value measured through the disposable blood glucose meter (not shown).

Up to now, the operations of the electronic device 200 according to the disclosure to correct the blood glucose information measured by the blood glucose meter 100 based on the predictive temperature of the inner skin part have been described in detail. Hereinafter, a method for correcting the blood glucose information measured by the blood glucose meter 100 in the electronic device 200 according to the disclosure will be described in detail.

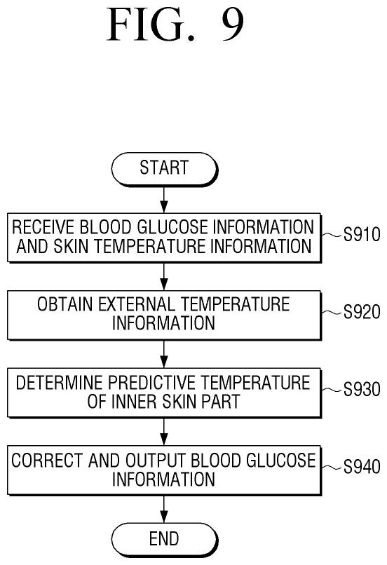

FIG. 9 is a flowchart of a method for correcting blood glucose information in the electronic device according to an embodiment of the disclosure.

As illustrated in FIG. 9, the electronic device 200 receives blood glucose information and skin temperature information from the blood glucose meter 100 (S910). Specifically, the blood glucose meter 100 determines a current value corresponding to a blood glucose value measured through an enzyme sensor inserted into a skin. In addition, the blood glucose meter 100 obtains skin temperature information sensed through a temperature sensor at the time when the blood glucose value is measured through the enzyme sensor. Thereafter, the blood glucose meter 100 transmits to the electronic device 200 the blood glucose information including the current value corresponding to the measured blood glucose value and the skin temperature information sensed at the time when the blood glucose value is measured.

When the blood glucose information and the skin temperature information are received, the electronic device 200 obtains external temperature information of a region in which a user wearing the blood glucose meter is located (S920).

According to an embodiment, the electronic device 200 may obtain a temperature value sensed by a temperature sensor included in the electronic device 200 as the external temperature information of the region in which the user wearing the blood glucose meter 100 is located.

According to another embodiment, the electronic device 200 may receive and obtain external temperature information from the blood glucose meter 100.

According to another embodiment, the electronic device 200 may receive and obtain external temperature information from a peripheral device 300 that is located within a region in which the user wearing the blood glucose meter 100 is located, among a plurality of peripheral devices 300 that are communicable and capable of sensing an external temperature.

When the external temperature information is obtained through the various embodiments, the electronic device 200 determines a predictive temperature of an inner skin part at which the enzyme sensor of the blood glucose meter 100 is located, using the skin temperature information received from the blood glucose meter 100 and the obtained external temperature information (S930).

Thereafter, the electronic device 200 corrects the blood glucose information received from the blood glucose meter 100 based on the determined predictive temperature and outputs the corrected blood glucose information (S940).

Hereinafter, a method for determining a predictive temperature of an inner skin part into which the enzyme sensor of the blood glucose meter 100 is inserted in the electronic device 200 according to the disclosure will be described in detail.

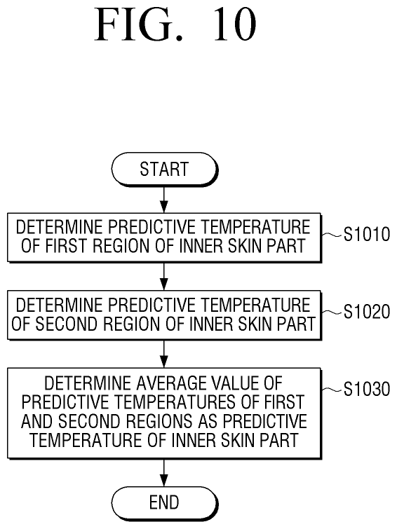

FIG. 10 is a flowchart of a method for determining a predictive temperature of an inner skin part into which the enzyme sensor of the blood glucose meter is inserted in the electronic device according to an embodiment of the disclosure.

As illustrated in FIG. 10, the electronic device 200 determines a predictive temperature of the first region of the inner skin part (S1010). Here, the first region of the inner skin part may be a region in which the enzyme sensor of the blood glucose meter 100 is inserted into a skin.

Thereafter, the electronic device 200 determines a predictive temperature of the second region of the inner skin part (S1020). Here, the second region of the inner skin part may be a region other than the first region in the region from a skin surface to a point at which deep part temperature information is measured, and the deep part temperature information may be a standard temperature of a body, for example 36.5° C.

Subsequently, the electronic device 200 determines an average value of the predictive temperature of the first region of the inner skin part and the predictive temperature of the second region of the inner skin part as the predictive temperature of the inner skin part into which the enzyme sensor of the blood glucose meter 100 is inserted (S1030).

Specifically, the electronic device 200 determines the predictive temperature of the first region based on the skin temperature information received from the blood glucose meter 100 and a thermal diffusion table that is based on a length of the enzyme sensor of the blood glucose meter inserted into a skin. The predictive temperature of the first region may be calculated based on the aforementioned Equation 1.

Before determining the value calculated based on Equation 1 as the predictive temperature of the first region, the electronic device 200 determines a predictive temperature of an outer skin part, and compares the determined predictive temperature of the outer skin part and the skin temperature information received from the blood glucose meter 100. Here, the predictive temperature of the outer skin part is a predictive temperature based on the obtained external temperature information and a time of exposure to an external temperature, and may be calculated from the aforementioned Equation 2.

As a result of comparison, if the predictive temperature of the outer skin part and the skin temperature information received from the blood glucose meter 100 are different, the electronic device 200 may determine the skin temperature information received from the blood glucose meter 100 as the predictive temperature of the first region of the inner skin part.

If the predictive temperature of the outer skin part and the skin temperature information received from the blood glucose meter 100 are not different, the electronic device 200 may determine the value calculated based on the aforementioned Equation 1 as the predictive temperature of the first region of the inner skin part.

In addition, the electronic device 200 may determine the predictive temperature of the second region based on the deep part temperature information, and a thermal diffusion table that is based on a distance from a skin surface to a point at which the deep part temperature information is measured and a length of the enzyme sensor 110-1. The predictive temperature of the second region may be calculated from the aforementioned Equation 3. Therefore, the electronic device 200 may determine a value calculated from Equation 3 as the predictive temperature of the second region.

When the predictive temperatures of the first and second regions of the inner skin part are determined, the electronic device 200 may determine an average value of the predictive temperatures of the first and second regions as the predictive temperature of the inner skin part into which the enzyme sensor of the blood glucose meter 100 is inserted.

Meanwhile, once the average value of the predictive temperatures of the first and second regions is calculated, the electronic device 200 may apply a preset weight to the calculated average value to determine the predictive temperature of the inner skin part into which the enzyme sensor of the blood glucose meter 100 is inserted.

Hereinafter, a method for setting a weight to be applied to the average value of the predictive temperatures of the first and second regions of the inner skin part in the electronic device according to the disclosure will be described in detail.

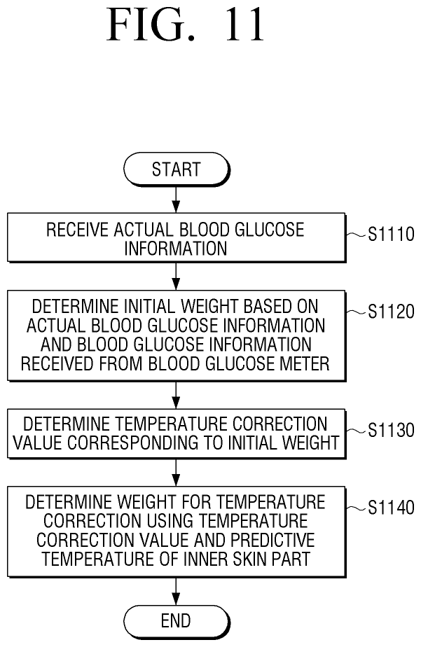

FIG. 11 is a flowchart of a method for setting a weight used to determine a predictive temperature of an inner skin part into which an enzyme sensor of the blood glucose meter is inserted in the electronic device according to an embodiment of the disclosure.

As illustrated in FIG. 11, the electronic device 200 receives actual blood glucose information measured by a disposable blood glucose meter (not shown), which is an external device (S1110). Here, the actual blood glucose information may be information measured under the same conditions as the blood glucose information received from the blood glucose meter (100). At this point, the same conditions may be a time and a place at which blood glucose is measured by the disposable blood glucose meter (not shown) and by the blood glucose meter 100.

Thereafter, the electronic device 200 determines an initial weight by using the actual blood glucose information received from the disposable blood glucose meter (not shown) and the blood glucose information received from the blood glucose meter 100 (S1120).

Specifically, the electronic device 200 may determine a value obtained by dividing a blood glucose value included in the actual blood glucose information by a blood glucose value included in the blood glucose information received from the blood glucose meter 100 as the initial weight.

Thereafter, the electronic device 200 obtains a temperature correction value corresponding to the initial weight with reference to a predefined temperature correction table (S1130). Subsequently, the electronic device 200 determines a weight for temperature correction using the temperature correction value and the predictive temperature of the inner skin part determined through the embodiment described above. Specifically, the electronic device 200 may determine a value obtained by dividing the temperature correction value by the predictive temperature of the inner skin part as the weight for temperature correction.

Thereafter, the electronic device 200 may finally determine the predictive temperature of the inner skin part by multiplying the average value of the predictive temperatures of the first and second regions of the inner skin part calculated through the aforementioned Equations 1 to 3 by the weight β for temperature correction.

Meanwhile, the electronic device control method according to the various embodiments described above may be coded in software and stored in a non-transitory readable medium. Such a non-transitory readable medium may be mounted and used in a variety of devices.

The non-transitory readable medium means a medium that is readable by a device and stores data semipermanently, rather than a medium that stores data for an instant, such as a register, cache or memory. Specifically, the non-transitory readable medium may be a CD, a DVD, a hard disk, a Blu-ray disk, a USB, a memory card, a ROM, or the like.

In addition, although preferred embodiments of the disclosure have been illustrated and described hereinabove, the disclosure is not limited to the particular embodiments described above, but may be variously modified by those skilled in the art to which the disclosure pertains without departing from the gist of the disclosure as claimed in the accompanying claims. These modifications should not be separately understood from the technical spirit and prospect of the disclosure.

1. An electronic device control method, comprising:
receiving blood glucose information and skin temperature information from a blood glucose meter;
obtaining external temperature information of a region in which a user wearing the blood glucose meter is located;
determining, by using the skin temperature information and the external temperature information, a predictive temperature of an inner skin part at which an enzyme sensor of the blood glucose meter is located;
correcting the blood glucose information on the basis of the determined predictive temperature; and
outputting the corrected blood glucose information.

2. The electronic device control method as claimed in claim 1, wherein the determining includes:
determining a predictive temperature of a first region of the inner skin part based on the skin temperature information and the external temperature information;
determining a predictive temperature of a second region of the inner skin part based on preset deep part temperature information; and
determining the predictive temperature of the inner skin part based on an average value of the predictive temperatures of the first and second regions.

3. The electronic device control method as claimed in claim 2, wherein in the determining of the predictive temperature of the first region, the predictive temperature of the first region is determined based on the skin temperature information and a thermal diffusion table that is based on a length of the enzyme sensor of the blood glucose meter inserted into a skin.

4. The electronic device control method as claimed in claim 2, wherein in the determining of the predictive temperature of the first region, the skin temperature information is compared with a predictive temperature that is calculated based on the external temperature information and a time of exposure to an external temperature, and if the two temperatures are different, the skin temperature information is determined as the predictive temperature of the inner skin part.

5. The electronic device control method as claimed in claim 2, wherein in the determining of the predictive temperature of the second region, the predictive temperature of the second region is determined based on the deep part temperature information, and a thermal diffusion table that is based on a distance from a skin surface to a point at which the deep part temperature information is measured and a length of the enzyme sensor.

6. The electronic device control method as claimed in claim 2, wherein the determining further includes determining a weight based on actual blood glucose information and the received blood glucose information, and
in the determining of the predictive temperature of the inner skin part, the predictive temperature of the inner skin part is determined by applying the weight to the average value of the predictive temperatures of the first and second regions.

7. The electronic device control method as claimed in claim 6, wherein the determining of the weight includes:
receiving actual blood glucose information measured under the same conditions as the received blood glucose information from an external device;
determining an initial weight using the actual blood glucose information and the received blood glucose information;
obtaining a temperature correction value corresponding to the initial weight with reference to a predefined temperature correction table; and
determining the weight for temperature correction using the temperature correction value and the predictive temperature of the inner skin part.

8. The electronic device control method as claimed in claim 2, wherein the external temperature information is sensed by at least one of an electronic device, a peripheral device that is communicable with the electronic device, or the blood glucose meter.

9. An electronic device, comprising:
a communicator;
an output unit; and
a processor configured to:
receive blood glucose information and skin temperature information from a blood glucose meter through the communicator,
obtain external temperature information of a region in which a user wearing the blood glucose meter is located,
determine, by using the skin temperature information and the external temperature information, a predictive temperature of an inner skin part at which an enzyme sensor of the blood glucose meter is located, and
correct the blood glucose information on the basis of the determined predictive temperature and control the output unit to output the corrected blood glucose information.

10. The electronic device as claimed in claim 9, wherein the processor determines a predictive temperature of a first region of the inner skin part based on the skin temperature information and the external temperature information, determines a predictive temperature of a second region of the inner skin part based on preset deep part temperature information, and determines the predictive temperature of the inner skin part based on an average value of the predictive temperatures of the first and second regions.

11. The electronic device as claimed in claim 10, wherein the processor determines the predictive temperature of the first region based on the skin temperature information and a thermal diffusion table that is based on a length of the enzyme sensor of the blood glucose meter inserted into a skin.

12. The electronic device as claimed in claim 10, wherein the processor compares the skin temperature information with a predictive temperature that is calculated based on the external temperature information and a time of exposure to an external temperature, and if the two temperatures are different, determines the skin temperature information as the predictive temperature of the inner skin part.

13. The electronic device as claimed in claim 10, wherein the processor determines the predictive temperature of the second region based on the deep part temperature information, and a thermal diffusion table that is based on a distance from a skin surface to a point at which the deep part temperature information is measured and a length of the enzyme sensor.

14. The electronic device as claimed in claim 10, wherein the processor determines a weight based on actual blood glucose information and the received blood glucose information, and determines the predictive temperature of the inner skin part by applying the weight to the average value of the predictive temperatures of the first and second regions.

15. The electronic device as claimed in claim 14, wherein when receiving actual blood glucose information measured under the same conditions as the received blood glucose information from an external device, the processor determines an initial weight using the actual blood glucose information and the blood glucose information, obtains a temperature correction value corresponding to the initial weight with reference to the predefined temperature correction table, and determines the weight for temperature correction using the temperature correction value and the predictive temperature of the inner skin part.
